(19) United States
(12) Patent Application Publication
(10) Pub. No.: US 2018/0199798 A1
(43) Pub. Date: Jul. 19, 2018

(54) OPTICAL SCANNING OBSERVATION SYSTEM

(71) Applicant: OLYMPUS CORPORATION, Tokyo (JP)

(72) Inventor: Kazuma Kaneko, Tokyo (JP)

(73) Assignee: OLYMPUS CORPORATION, Tokyo (JP)

(21) Appl. No.: 15/920,550

(22) Filed: Mar. 14, 2018

Related U.S. Application Data
(63) Continuation of application No. PCT/JP2016/053659, filed on Feb. 8, 2016.

(30) Foreign Application Priority Data
Sep. 17, 2015 (JP) 2015-184108

(51) Int. Cl. A61B 1/00; A61B 1/07; A61B 1/04; G06T 3/60; G06T 3/40; G02B 26/10

(52) U.S. Cl. CPC A61B 1/00183; A61B 1/07; A61B 1/04; G02B 26/103; G06T 3/60; G06T 3/40; A61B 1/00009

(57) ABSTRACT

An optical scanning observation system includes: a light-guide that guides illumination light; an actuator that causes the end portion of the light-guide to oscillate, to thereby be capable of shifting an irradiation position of the illumination light emitted to an object; a light detection section that generates a light detection signal based on return light from the object, and output the generated light detection signal; an error angle acquisition section that acquires an error angle indicating a degree of deviation of the irradiation position of the illumination light; and an image generation section that generates a rotated image by rotating pixel information acquired by converting the light detection signal outputted from the light detection section by an angle acquired by subtracting the error angle from a desired angle of rotation.

### CROSS REFERENCE TO RELATED APPLICATION

This application is a continuation application of PCT/JP2016/053659 filed on Feb. 8, 2016 and claims benefit of Japanese Application No. 2015-184108 filed in Japan on Sep. 17, 2015, the entire contents of which are incorporated herein by this reference.

### BACKGROUND OF INVENTION

### 1. Field of the Invention

The present invention relates to an optical scanning observation system, and more particularly to an optical scanning observation system that scans an object to acquire an image.

### 2. Description of the Related Art

Various kinds of techniques have been proposed for endoscopes in medical fields, for reducing a diameter size of an insertion portion to be inserted into a body cavity of a subject to be examined in order to reduce a burden on the subject to be examined. As one example of such techniques, a scanning endoscope which does not include a solid-state image pickup device in a part corresponding to the above-described insertion portion is known.

Specifically, a system including a scanning endoscope is, for example, configured to transmit illumination light emitted from a light source by an illumination optical fiber, two-dimensionally scan an object along a predetermined scanning path by driving an actuator for oscillating a distal end portion of the illumination optical fiber, receive return light from the object by a light-receiving optical fiber, and generate an image of the object based on the return light received by the light-receiving optical fiber. Japanese Patent Application Laid-Open Publication No. 2011-115252, for example, discloses a medical observation system having a configuration similar to the above-described configuration.

### SUMMARY OF THE INVENTION

An optical scanning observation system according to one aspect of the present invention includes: a light-guide configured to guide illumination light supplied from a light source unit, and emit the illumination light from an end portion of the light-guide; an actuator configured to cause the end portion of the light-guide to oscillate, to thereby be capable of shifting, along a spiral-shaped scanning path, an irradiation position of the illumination light emitted to an object through the end portion; a light detection section configured to detect return light from the object, generate a light detection signal based on the detected return light, and output the generated light detection signal; an error angle acquisition section configured to perform processing for acquiring an error angle indicating a degree of deviation of the irradiation position of the illumination light, the irradiation position corresponding to an outermost point of the spiral-shaped scanning path; and an image generation section configured to perform processing for generating a rotated image by rotating pixel information acquired by converting the light detection signal outputted from the light detection section by an angle acquired by subtracting the error angle from a desired angle of rotation with a center point of the spiral-shaped scanning path as a rotation center.

### BRIEF DESCRIPTION OF THE DRAWINGS

FIG. 1 illustrates a configuration of a main part of an optical scanning observation system according to an embodiment.

FIG. 2 is a cross-sectional view for describing a configuration of an actuator.

FIG. 3 illustrates one example of signal waveforms of drive signals supplied to the actuator.

FIG. 4 illustrates one example of a spiral-shaped scanning path from a center point A to an outermost point B.

FIG. 5 illustrates one example of a spiral-shaped scanning path from the outermost point B to the center point A.

FIG. 6 illustrates one example of a configuration of an image generation section.

FIG. 7 illustrates one example of an object to be scanned by an endoscope.

FIG. 8 illustrates one example of an original image generated when the object in FIG. 7 is scanned.

FIG. 9 illustrates one example of a rotated image generated by using the original image in FIG. 8.

FIG. 10 illustrates processing related to a calculation of an error angle θe.

### DETAILED DESCRIPTION OF THE PREFERRED EMBODIMENT(S)

Hereinafter, an embodiment of the present invention will be described with reference to drawings.

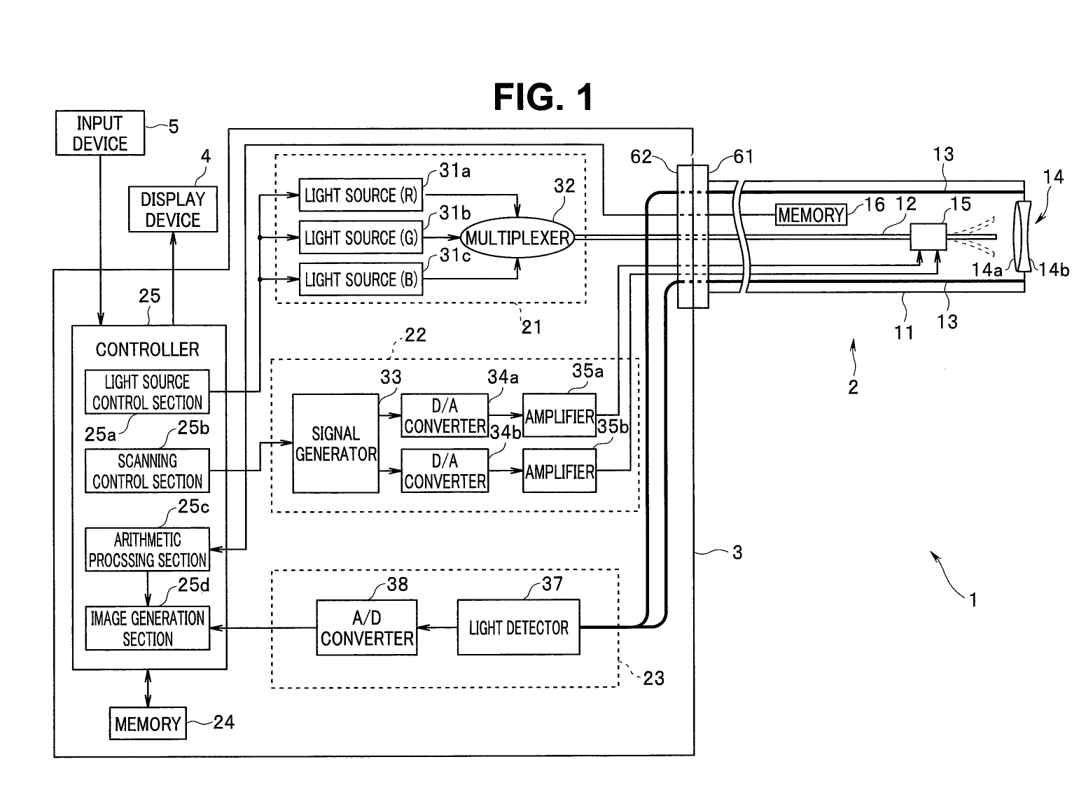

FIGS. 1 to 10 relate to the embodiment of the present invention. FIG. 1 illustrates a configuration of a main part of an optical scanning observation system according to the embodiment.

As shown in FIG. 1, for example, an optical scanning observation system 1 includes a scanning endoscope 2 configured to be inserted into a body cavity of a subject to be examined, a main body apparatus 3 to which the endoscope 2 is connectable, a display device 4 that is connected to the main body apparatus 3, and an input device 5 configured to be capable of inputting information and giving an instruction to the main body apparatus 3.

The endoscope 2 includes an insertion portion 11 formed in an elongated shape insertable into a body cavity of a subject to be examined.

The insertion portion 11 includes at a proximal end portion thereof a connector portion 61 for detachably connecting the endoscope 2 to a connector receiving portion 62 of the main body apparatus 3.

Inside the connector portion 61 and the connector receiving portion 62, electric connector devices, not shown, for electrically connecting the endoscope 2 and the main body apparatus 3 are respectively provided. In addition, inside the connector portion 61 and the connector receiving portion 62, optical connector devices, not shown, for optically connecting the endoscope 2 and the main body apparatus 3 are respectively provided.

An illumination fiber 12, which is an optical fiber that guides illumination light supplied from a light source unit 21 of the main body apparatus 3 to emit the guided illumination light from an emission end portion thereof, and a light-receiving fiber 13 including one or more optical fibers for receiving return light from an object to guide the received return light to a detection unit 23 of the main body apparatus 3 are inserted in a part from the proximal end portion to the distal end portion inside the insertion portion 11. That is, the illumination fiber 12 includes a function as a light-guide.

An incident end portion including a light incident surface of the illumination fiber 12 is arranged at a multiplexer 32 disposed inside the main body apparatus 3. In addition, the emission end portion including a light emission surface of the illumination fiber 12 is arranged in the vicinity of a light incident surface of a lens 14a provided at the distal end portion of the insertion portion 11.

An incident end portion including a light incident surface of the light-receiving fiber 13 is arranged so as to be fixed around the light emission surface of the lens 14b on the distal end surface of the distal end portion of the insertion portion 11. Furthermore, an emission end portion including a light emission surface of the light-receiving fiber 13 is arranged at a light detector 37 disposed inside the main body apparatus 3.

The illumination optical system 14 includes the lens 14a on which the illumination light passed through the light emission surface of the illumination fiber 12 is incident, and the lens 14b from which the illumination light passed through the lens 14a is applied to the object.

An actuator 15, which is driven based on a drive signal supplied from a driver unit 22 of the main body apparatus 3, is provided at the halfway portion of the illumination fiber 12 on the distal end portion side of the insertion portion 11.

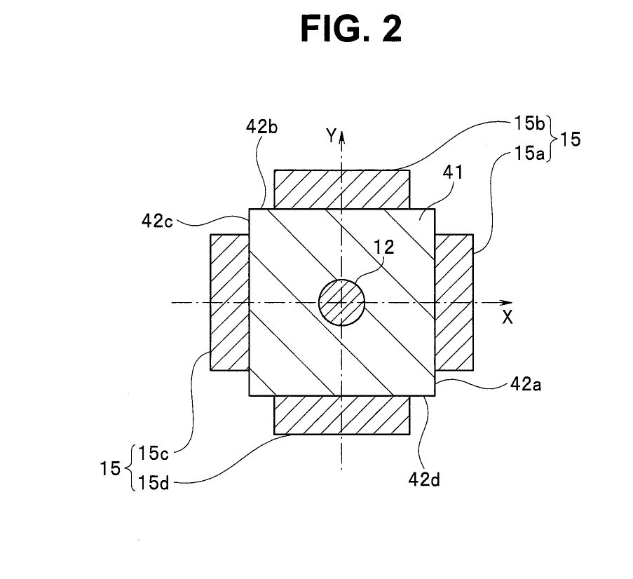

The illumination fiber 12 and the actuator 15 are respectively arranged so as to have a positional relationship shown in FIG. 2, for example, on the cross section vertical to a longitudinal axis direction of the insertion portion 11. FIG. 2 is a cross-sectional view for describing the configuration of the actuator.

As shown in FIG. 2, a ferrule 41 as a joining member is arranged between the illumination fiber 12 and the actuator 15. Specifically, the ferrule 41 is made of zirconia (ceramic) or nickel, for example.

As shown in FIG. 2, the ferrule 41 is formed as a quadrangular prism, and includes side surfaces 42a and 42c vertical to an X-axis direction which is a first axis direction perpendicular to the longitudinal axis direction of the insertion portion 11, and side surfaces 42b and 42d vertical to a Y-axis direction which is a second axis direction perpendicular to the longitudinal axis direction of the insertion portion 11. In addition, the illumination fiber 12 is arranged so as to be fixed at the center of the ferrule 41.

As shown in FIG. 2, for example, the actuator 15 includes a piezoelectric element 15a arranged along the side surface 42a, a piezoelectric element 15b arranged along the side surface 42b, a piezoelectric element 15c arranged along the side surface 42c, and a piezoelectric element 15d arranged along the side surface 42d.

Each of the piezoelectric elements 15a to 15d has a polarization direction individually set in advance, and is configured to expand and contract according to the driving voltage applied based on the drive signal supplied from the main body apparatus 3.

That is, the piezoelectric elements 15a and 15c of the actuator 15 are configured as an x-axis actuator that vibrates in response to the drive signal supplied from the main body apparatus 3, to thereby enable the illumination fiber 12 to oscillate in the x-axis direction. In addition, the piezoelectric elements 15b and 15d of the actuator 15 are configured as a y-axis actuator that vibrates in response to the drive signal supplied from the main body apparatus 3, to thereby enable the illumination fiber 12 to oscillate in the y-axis direction.

The insertion portion 11 includes inside thereof a non-volatile memory 16 in which, for example, information on an error angle θe to be used in processing to be described later is stored as endoscope information specific to each endoscope 2. The endoscope information stored in the memory 16 is read by a controller 25 of the main body apparatus 3, when the connector portion 61 of the endoscope 2 and the connector receiving portion 62 of the main body apparatus 3 are connected to each other and the power source of the main body apparatus 3 is turned on.

The main body apparatus 3 includes a light source unit 21, a driver unit 22, the detection unit 23, a memory 24, and the controller 25.

The light source unit 21 includes a light source 31a, a light source 31b, a light source 31c, and the multiplexer 32.

The light source 31a includes a laser light source, etc., for example. The light source 31a is configured to emit light of red wavelength band (hereinafter, also referred to as R-light) to the multiplexer 32, when turned on by the control of the controller 25.

The light source 31b includes a laser light source, etc., for example. The light source 31b is configured to emit light of green wavelength band (hereinafter, also referred to as G-light) to the multiplexer 32, when turned on by the control of the controller 25.

The light source 31c includes a laser light source, etc., for example. The light source 31c is configured to emit light of blue wavelength band (hereinafter, also referred to as B-light) to the multiplexer 32, when turned on by the control of the controller 25.

The multiplexer 32 is configured to be capable of multiplexing the R-light emitted from the light source 31a, the G-light emitted from the light source 31b, and the B-light emitted from the light source 31c, and supplying the multiplexed light to the light incident surface of the illumination fiber 12.

The driver unit 22 is configured to generate and supply a drive signal DA for driving the x-axis actuator of the actuator 15 based on the control by the controller 25. In addition, the driver unit 22 is configured to generate and supply a drive signal DB for driving the y-axis actuator of the actuator 15 based on the control by the controller 25. Furthermore, the driver unit 22 includes a signal generator 33, D/A converters 34a and 34b, and amplifiers 35a and 35b.

The signal generator 33 is configured to generate a signal having a waveform expressed by an equation (1) shown below, for example, as a first drive control signal for causing the emission end portion of the illumination fiber 12 to oscillate in the x-axis direction, to output the generated signal to the D/A converter 34a, based on the control by the controller 25. Note that, in the equation (1) below, X(t) represents a signal level at a time t, Ax represents an amplitude value which is not dependent on the time t, and G(t) represents a predetermined function to be used in modulation of a sine wave sin (2πft).

X(t)=Ax×G(t)×sin(2πft) (1)

In addition, the signal generator 33 is configured to generate a signal having a waveform expressed by an equation (2) shown below, for example, as a second drive control signal for causing the emission end portion of the illumination fiber 12 to oscillate in the y-axis direction, to output the generated signal to the D/A converter 34b, based on the control by the controller 25. Note that, in the equation (2) below, Y(t) represents a signal level at the time t, Ay represents an amplitude value which is not dependent on the time t, G(t) represents a predetermined function to be used in modulation of a sine wave sin (2πft+φ), and φ represents a phase.

Y(t)=Ay×G(t)×sin(2πft+φ) (2)

The D/A converter 34a is configured to convert the first drive control signal, which is a digital signal, outputted from the signal generator 33 into the drive signal DA, which is an analog voltage signal, and output the drive signal DA to the amplifier 35a.

The D/A converter 34b is configured to convert the second drive control signal, which is a digital signal, outputted from the signal generator 33 into the drive signal DB, which is an analog voltage signal, and output the drive signal DB to the amplifier 35b.

The amplifier 35a is configured to amplify the drive signal DA outputted from the D/A converter 34a, to output the amplified drive signal DA to the piezoelectric elements 15a and 15c of the actuator 15.

The amplifier 35b is configured to amplify the drive signal DB outputted from the D/A converter 34b, to output the amplified drive signal DB to the piezoelectric elements 15b and 15d of the actuator 15.

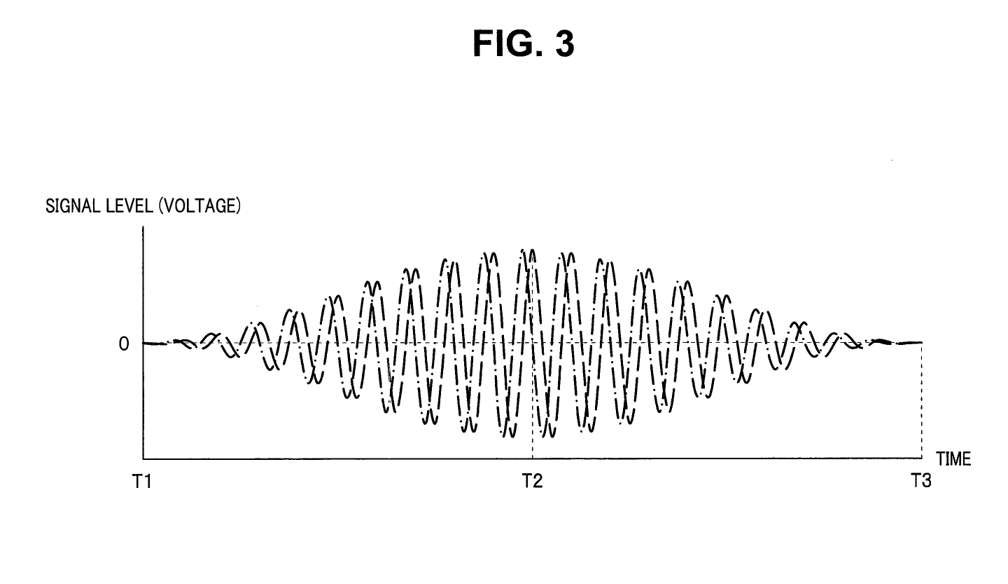

When Ax is set to be equal to Ay and φ is set to be equal to π/2 in the equations (1) and (2), for example, the driving voltage according to the drive signal DA having the signal waveform as shown by the dashed line in FIG. 3 is applied to the piezoelectric elements 15a and 15c of the actuator 15, and the driving voltage according to the drive signal DB having the signal waveform as shown by the one-dot chain line in FIG. 3 is applied to the piezoelectric elements 15b and 15d of the actuator 15. FIG. 3 illustrates one example of the signal waveforms of the drive signals supplied to the actuator.

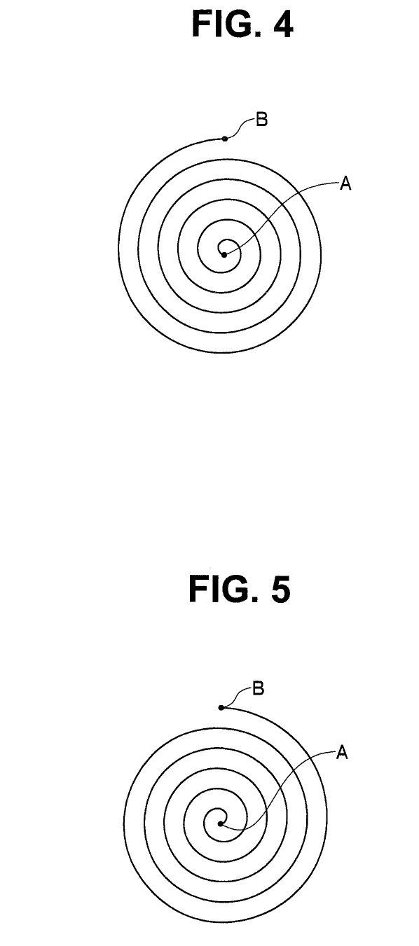

In addition, when the driving voltage according to the drive signal DA having the signal waveform shown by the dashed line in FIG. 3 is applied to the piezoelectric elements 15a and 15c of the actuator 15 and the driving voltage according to the drive signal DB having the signal waveform shown by the one-dot chain line in FIG. 3 is applied to the piezoelectric elements 15b and 15d of the actuator 15, for example, the emission end portion of the illumination fiber 12 is oscillated spirally, and the surface of the object is scanned in accordance with the oscillation along the spiral-shaped scanning path as shown in FIGS. 4 and 5. FIG. 4 illustrates one example of the spiral-shaped scanning path from a center point A to an outermost point B. FIG. 5 illustrates one example of the spiral-shaped scanning path from the outermost point B to the center point A.

Specifically, at the time T1, the illumination light is applied to the position corresponding to the center point A of the irradiation position of the illumination light on the surface of the object. After that, as the signal levels (voltages) of the drive signals DA and DB increase from the time T1 to the time T2, the irradiation position of the illumination light on the surface of the object is shifted toward the outside so as to draw a first spiral-shaped scanning path, with the center point A as the starting point. Then, when the time reaches the time T2, the illumination light is applied to the outermost point B of the irradiation position of the illumination light on the surface of the object. As the signal levels (voltages) of the drive signals DA and DB decrease from the time T2 to the time T3, the irradiation position of the illumination light on the surface of the object is shifted toward the inside so as to draw a second spiral-shaped scanning path, with the outermost point B as the starting point. Then, when the time reaches the time T3, the illumination light is applied to the center point A on the surface of the object.

That is, the actuator 15 causes the emission end portion of the illumination fiber 12 to oscillate based on the drive signals DA and DB supplied from the driver unit 22, thereby capable of shifting the irradiation position of the illumination light emitted to the object through the emission end portion, along the spiral-shaped scanning path shown in FIGS. 4 and 5.

The detection unit 23 has a function as a light detection section, and configured to detect, in succession, the return light received by the light-receiving fiber 13 of the endoscope 2 and generate light detection signals according to the intensities of the return light detected in succession, to sequentially output the generated light detection signals. Specifically, the detection unit 23 includes a light detector 37 and the A/D converter 38.

The light detector 37 includes an avalanche photodiode, for example, and configured to detect, in succession, the light (return light) emitted from the light emission surface of the light-receiving fiber 13, generate analog light detection signals according to the intensities of the light detected in succession, to sequentially output the generated light detection signals to the A/D converter 38.

The A/D converter 38 is configured to convert the analog light detection signals outputted from the light detector 37 into the digital light detection signals, to sequentially output the digital light detection signals to the controller 25.

The memory 24 stores, as the control information to be used for controlling the main body apparatus 3, information such as parameters for identifying the signal waveforms in FIG. 3 and a mapping table which is a table for showing the correspondence relation between the output timings of the light detection signals sequentially outputted from the detection unit 23 and pixel positions to which pieces of the pixel information acquired by converting the light detection signals are applied, for example.

The controller 25 includes an integrated circuit such as FPGA (Field Programmable Gate Array), for example, and is configured to be capable of performing an action in response to the operation of the input device 5.

The controller 25 detects the connection state of the connector 61 to the connector receiving portion 62 through a signal line or the like, not shown, to thereby capable of detecting whether the insertion portion 11 is electrically connected to the main body apparatus 3. Specifically, the controller 25 measures, for example, a resistance value of a resistor provided at a predetermined terminal of the connector portion 61, or a potential difference at a predetermined terminal of the connector receiving portion 62 as a connecting destination of the GND terminal of the connector portion 61, to thereby detect whether the insertion portion 11 is electrically connected to the main body apparatus 3.

Note that when the above-described resistance value or the potential difference is measured, it is preferable to set a detection period of about 0.5 seconds, for example, in order to prevent chattering. In addition, when the controller 25 fails to measure the resistance value or the potential difference, for example, the controller 25 may perform an action for informing the failure in the detection of the connection of the insertion portion 11 to the main body apparatus 3 or an action for informing a request for cleaning the connector portion 61 and the connector receiving portion 62.

The controller 25 is configured to be capable of reading the control information stored in the memory 24 and performing an action in accordance with the read control information, when the power source of the main body apparatus 3 is turned on. In addition, the controller 25 includes a light source control section 25a, a scanning control section 25b, an arithmetic processing section 25c, and an image generation section 25d.

The light source control section 25a is configured to perform control on the light source unit 21 for causing the light source unit 21 to repeatedly emit the R-light, the G-light, and the B-light in this order, for example, based on the control information read from the memory 24.

The scanning control section 25b is configured to perform control on the driver unit 22 for causing the driver unit 22 to generate the drive signals having the signal waveforms as shown in FIG. 3, for example, based on the control information read from the memory 24.

The arithmetic processing section 25c is configured to perform arithmetic processing of rotating the pixel positions, which are in the unrotated state and specified in the mapping table included in the control information read from the memory 24, with the center point A of the spiral-shaped scanning path as the rotation center, based on an angle of rotation θi set in response to the operation of the input device 5 and the error angle θe included in the endoscope information read from the memory 16, to thereby acquire pixel positions after the rotation, and output the pixel positions after the rotation acquired by the arithmetic processing to the image generation section 25d.

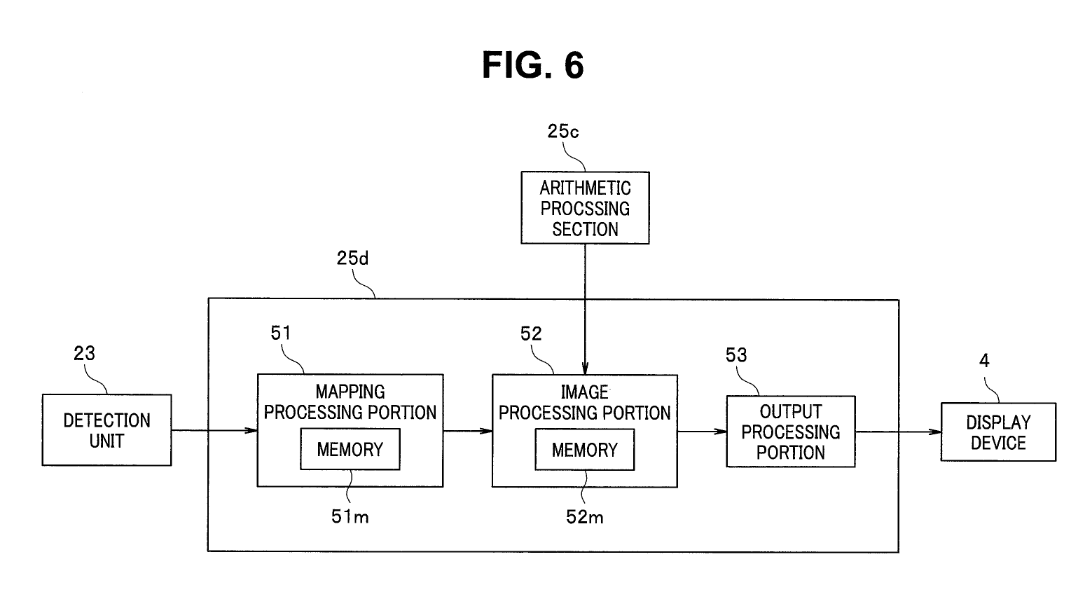

The image generation section 25d is configured to convert the light detection signals, which are sequentially outputted from the detection unit 23 within the period from the time T1 to the time T2, into the pieces of pixel information such as RGB components, to map (arrange) the pieces of pixel information, based on the mapping table included in the control information read from the memory 24, and generate, for each frame, an original image, which is the image before being rotated according to the angle of rotation θi and the error angle θe, with the center point A of the spiral-shaped scanning path as the rotation center. In addition, the image generation section 25d is configured to remap (rearrange) the pieces of pixel information in the respective pixel positions in the original image generated as described above in accordance with the respective pixel positions after the rotation, which are outputted from the arithmetic processing section 25c, to thereby generate, for each frame, a rotated image, which is an image after being rotated according to the angle of rotation θi and the error angle θe, with the center point A of the spiral-shaped scanning path as the rotation center, and output an observation image based on the generated rotated image to the display device 4. Furthermore, the image generation section 25d includes a mapping processing portion 51, an image processing portion 52, and an output processing portion 53, as shown in FIG. 6, for example. FIG. 6 illustrates one example of the configuration of the image generation section.

The mapping processing portion 51 includes a memory 51m having a capacity capable of storing the image for at least one frame. In addition, the mapping processing portion 51 is configured to perform the mapping processing for converting the light detection signals sequentially outputted from the detection unit 23 within the period from the time T1 to the time T2 into the pieces of pixel information and mapping (arranging) the pieces of pixel information, based on the mapping table included in the control information read from the memory 24, to thereby generate the original image for each frame, and sequentially write the original images thus generated in the memory 51m.

The image processing portion 52 includes a memory 52m having a capacity capable of storing the image for at least one frame. In addition, the image processing portion 52 is configured to read the original image for the latest one frame written in the memory 51m, to perform predetermined image processing on the read original image. Furthermore, the image processing portion 52 performs the remapping processing for remapping (rearranging) the pieces of pixel information in the respective pixel positions in the original image subjected to the predetermined image processing in accordance with the respective pixel positions after the rotation, which are outputted from the arithmetic processing section 25c, to thereby generate the rotated images for each frame, and write the rotated image thus generated in the memory 52m.

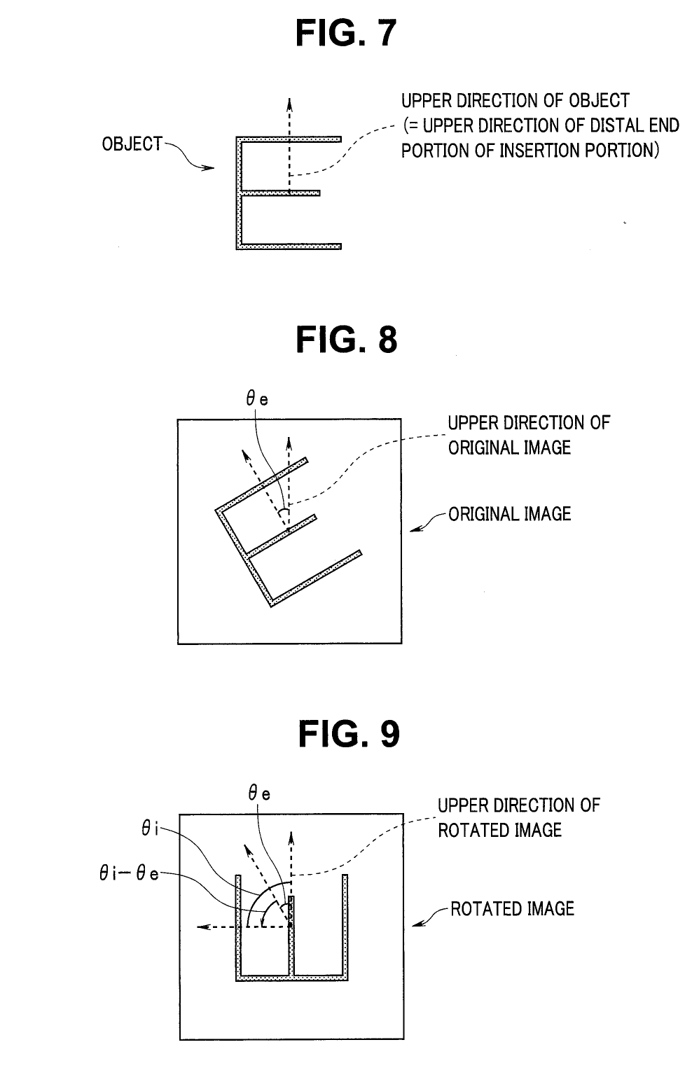

The endoscope 2 might be used in the state including a manufacturing error (manufacturing variation) of the actuator 15 due to the attaching position of the actuator 15 being deviated from the standard state, for example. When the character “E” as shown in FIG. 7 is scanned as an object, for example, the manufacturing error (manufacturing variation) of the actuator 15 might cause occurrence of the phenomenon in which the character “E” included in the original image generated by the mapping processing is rotated by the error angle θe, with the center point A of the spiral-shaped scanning path as the rotation center, irrespective of the angle of rotation θi set by the user (see FIG. 8). Therefore, in the present embodiment, as shown in FIG. 9, for example, the rotated image in the state where the above-described phenomenon has been solved is generated by taking the angle of rotation θi as well as the error angle θe into consideration. FIG. 7 illustrates one example of the object to be scanned by an endoscope. FIG. 8 illustrates one example of the original image generated when the object in FIG. 7 is scanned. FIG. 9 illustrates one example of the rotated image generated by using the original image in FIG. 8.

In addition, with the present embodiment, the manufacturing error (manufacturing variation) of the actuator 15 is manifested as the rotation error, with the center point A of the spiral-shaped scanning path as the rotation center, in the original image generated through the mapping processing. Therefore, the present embodiment is capable of preferably correcting the manufacturing error (manufacturing variation) of the actuator 15, which is manifested as the above-described rotation error.

In addition, the image processing portion 52 is configured to perform, as predetermined image processing, conversion processing for converting the RGB components of the original image read from the memory 51m into luminance components and color difference components, color correction processing for performing color correction processing using a predetermined matrix on the color difference components acquired through the conversion processing, enhancement processing for performing contour enhancement or structure enhancement on the luminance components acquired through the conversion processing, reconversion processing for reconverting the color difference components subjected to the color correction processing and the luminance components subjected to the enhancement processing into the RGB components, and gamma correction processing for performing a gamma correction on the RGB components acquired through the reconversion processing, for example.

Note that the predetermined image processing exemplified above is not limited to the processing to be performed on the original image read from the memory 51m, but may be processing to be performed on the rotated image generated by using the original image. In addition, in the present embodiment, for example, clip processing or chroma suppression processing, as processing for limiting the upper limit value of the signal value of the digital signal to a predetermined value smaller than the maximum value, may be incorporated in the predetermined image processing to be performed by the image processing portion 52 to prevent the phenomenon in which the part where halation occurs in the original image read from the memory 51m is colored with non-saturated color components.

The output processing portion 53 is configured to sequentially read, frame by frame, the rotated images written in the memory 52m, and perform predetermined processing such as trimming or masking on each of the read rotated images to generate a circular observation image. In addition, the output processing portion 53 is configured to output the observation image generated as described above to the display device 4 in compliance with the transmission standard of the digital video, such as HD-SDI method.

The display device 4 includes an LCD (liquid crystal display) which supports the digital input, for example, and is configured to be capable of displaying the observation image outputted from the main body apparatus 3.

The input device 5 includes switches, buttons, and the like, for example. Note that the input device 5 may be configured as a device separated from the main body apparatus 3, or as an interface integrated with the main body apparatus 3.

Next, description will be made on the working of the optical scanning observation system 1 having the configuration as described above. Note that description will be made hereinafter by taking, as an example, the case where the error angle θe and the angle of rotation θi are the angles with the center point A of the first spiral-shaped scanning path in FIG. 4 as the rotation center.

First, description will be made on a specific example of the acquiring method of the error angle θe to be stored in the memory 16.

When manufacturing the endoscope 2, for example, a factory worker connects the respective components of the optical scanning observation system 1 and turns on the power source of the system. Then, the worker arranges the light-receiving surface of the PSD (position sensitive device), which is not shown, and the distal end surface of the endoscope 2 so as to be opposed to each other and disposes the cable, etc., so that the output signal from the PSD is inputted to the arithmetic processing section 25c.

After that, the factory worker operates the scanning starting switch (not shown) of the input device 5, to give an instruction for starting the scanning by the endoscope 2 to the controller 25. In response to such an instruction, the light-receiving surface of the PSD is scanned along the spiral-shaped scanning path, and the output signals from the PSD are sequentially inputted to the arithmetic processing section 25c.

When detecting that the scanning starting switch of the input device 5 is operated and the error angle θe is not included in the endoscope information read from the memory 16, the arithmetic processing section 25c acquires a coordinate value MV corresponding to the outermost point B of the spiral-shaped scanning path shown in FIGS. 4 and 5, based on the output signals sequentially outputted from the PSD. In addition, the arithmetic processing section 25c having a function as an error angle acquisition section performs processing for calculating the error angle θe, based on the coordinate value MV acquired as described above and a coordinate value IV of the outermost point B, which is acquired when the light-receiving surface of the PSD is scanned with the endoscope 2 including the actuator 15 arranged in a standard arrangement state.

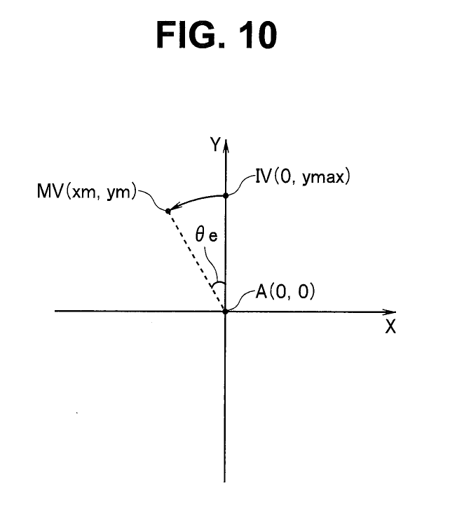

When the coordinate values acquired based on the output signals sequentially outputted from the PSD are coordinate values of an XY orthogonal coordinate system with the coordinate value of the center point A of the first spiral-shaped scanning path shown in FIG. 10, as an origin (0, 0), for example, a coordinate value MV (xm, ym) different for each endoscope 2 due to the manufacturing error (manufacturing variation) of the actuator 15 can be acquired and the coordinate value IV can be expressed as a coordinate value (0, ymax) on the Y axis. Therefore, in such a case, the angle of rotation of the coordinate value MV (xm, ym) with respect to the coordinate value IV (0, ymax) can be calculated as the error angle θe indicating the degree of deviation of the irradiation position of the illumination light, the irradiation position corresponding to the outermost point B of the first spiral-shaped scanning path. FIG. 10 illustrates processing related to a calculation of an error angle θe.

Note that the coordinate value IV may be included in advance in the control information read from the memory 24, for example, or may be inputted in response to the operation of the input device 5 as far as the coordinate value IV is handled as a known value when the error angle θe is calculated.

The arithmetic processing section 25c causes the memory 16 to store the error angle θe acquired as described above, and then performs control for displaying, on the display device 4, a character string, and the like, for informing the factory worker that the processing related to the acquisition of the error angle θe has been completed.

Note that, according to the present embodiment, as far as the angle of rotation of the coordinate value MV with respect to the coordinate value IV can be identified, another parameter other than the angle of rotation may be stored in the memory 16 as the error angle θe.

Next, description will be made on a specific example of the operation related to the generation of the rotated image based on the angle of rotation θi and the error angle θe.

The user such as an operator connects the respective components of the optical scanning observation system 1 and turns on the power source of the optical scanning observation system 1, and thereafter operates the scanning starting switch of the input device 5, to give the controller 25 an instruction to start the scanning by the endoscope 2. In addition, the user operates the input device 5 after starting the scanning by the endoscope 2, to give the controller 25 an instruction for setting the angle of rotation θi of the observation image displayed on the display device 4 to a desired angle of rotation.

Note that, in the present embodiment, for example, every time the image rotation button (not shown) provided on the input device 5 is pressed once, the angle of rotation θi may be changed by angles of 45 degrees (in the order of 0°→45°→90°→ . . . →315°→0°), the angle of rotation θi may be changed according to the rotation operation of a jog dial (not shown) provided on the input device 5, or the angle of rotation θi may be changed according to the touch operation of the tough panel (not shown) provided on the input device 5.

The arithmetic processing section 25c reads the control information stored in advance in the memory 24 and the endoscope information stored in advance in the memory 16, when the connector portion 61 of the endoscope 2 and the connector receiving portion 62 of the main body apparatus 3 are connected with each other and the power source of the main body apparatus 3 is turned on. In addition, when detecting that the scanning starting switch of the input device 5 is operated and the error angle θe is included in the endoscope information read from the memory 16, the arithmetic processing section 25c performs arithmetic processing for rotating the pixel positions, which are in the unrotated state and specified in the mapping table included in the control information read from the memory 24, with the center point A of the first spiral-shaped scanning path as the rotation center, based on the angle of rotation θi set according to the operation of the image rotation button of the input device 5 and the detected error angle θe, to acquire the pixel positions after the rotation.

Hereinafter, description will be made on a specific example of the arithmetic processing for acquiring the pixel positions after the rotation.

The arithmetic processing section 25c extracts, from the mapping table included in the control information read from the memory 24, the pixel at the pixel position corresponding to the center point A of the first spiral-shaped scanning path in FIG. 4, the pixel being the destination to which the pixel information acquired by converting the light detection signal outputted from the detection unit 23 at the output timing corresponding to the time T1 is applied. Then, the arithmetic processing section 25c including a function of a setting section performs the processing for setting the XY orthogonal coordinate system with the pixel of the rotation center as the origin (0, 0), for example, as the processing for setting the pixel extracted as described above as the pixel of the rotation center of the rotated image generated by the image generation section 25d.

The arithmetic processing section 25c performs processing for transforming the pixel position (PXA, PYA) before rotation, which is the pixel position specified in the mapping table included in the control information read from the memory 24, according to a predetermined transformation pattern, to thereby acquire the coordinate value (Pxa, Pya) of the XY orthogonal system set as described above, and thereafter transforming the acquired coordinate value (Pxa, Pya) into a coordinate value (Pr, Pθ) in the polar coordinate format.

The arithmetic processing section 25c performs processing for calculating the coordinate value (Pr, Pθ+(θi−θe)) in the polar coordinate format by adding the angle, which is acquired by subtracting the error angle θe from the angle of rotation θi, to Pθ of the coordinate value (Pr, Pθ) transformed as described above, and transforming the calculated coordinate value (Pr, Pθ+(θi−θe)) into the coordinate value (Pxb, Pyb) of the XY orthogonal coordinate.

Then, the arithmetic processing section 25c transforms the coordinate value (Pxb, Pyb) acquired as described above according to a pattern that is reversed from the above-described predetermined transformation pattern, to acquire the pixel position (PXB, PYB) after the rotation.

On the other hand, the mapping processing portion 51 performs the mapping processing for transforming the light detection signals sequentially outputted from the detection unit 23 within the period from the time T1 to the time T2 into the pieces of pixel information and mapping (arranging) the pieces of pixel information, based on the mapping table included in the control information read from the memory 24, to thereby generate the original image for each frame, and write the original images thus generated sequentially in the memory 51m.

The image processing portion 52 reads the original image for the latest one frame, which is written into the memory 51m, performs predetermined image processing on the read original image, and further performs remapping processing for remapping (rearranging) the pieces of pixel information in the respective pixel positions of the original image subjected to the predetermined image processing in accordance with the respective pixel positions after the rotation, which are outputted from the arithmetic processing section 25c, to thereby generate the rotated image for each frame, and write the rotated images thus generated sequentially into the memory 52m. That is, the image processing portion 52 generates each of the rotated images by rotating the pieces of pixel information in the respective pixel positions of the original image read from the memory 51m by the angle acquired by subtracting the error angle θe from the angle of rotation θi with the center point A of the first spiral-shaped scanning path as the rotation center, based on the pixel positions after the rotation outputted from the arithmetic processing section 25c.

The output processing portion 53 sequentially reads, frame by frame, the rotated images which are written into the memory 52m, to generate a circular observation image by performing predetermined processing such as trimming or masking on each of the read rotated images and output the generated observation image to the display device 4 in compliance with the transmission standard of the digital video.

As described above, the present embodiment enables the rotated image, which is rotated by the user's desired angle of rotation with the center point A of the spiral-shaped scanning path as the rotation center, to be displayed as the observation image on the display device 4, while removing the rotation of the image caused by the manufacturing error (manufacturing variation) of the actuator 15. Therefore, with the present embodiment, it is possible to reduce as much as possible the sense of visual discomfort that occurs when the image acquired by scanning the object is displayed on the display device.

In addition, with the present embodiment, a parameter for distortion correction acquired in advance for each endoscope 2 is used, for example, to perform distortion correction on the original image read from the memory 51m, and thereafter a rotated image can be generated. Therefore, the present embodiment is capable of suppressing as much as possible the distortion of the observation image, which might occur due to the generation of the rotated image based on the angle of rotation θi and the error angle θe.

Note that the image processing portion 52 according to the present embodiment may perform variable magnification processing which is processing for magnifying or reducing the original image read from the memory 51m, for example, in addition to the remapping processing.

In addition, the image generation section 25d according to the present embodiment may perform operation for causing the display device 4 to display visual information such as a character string and/or a mark indicating the current set value of the angle of rotation θi, for example, together with the observation image.

In addition, with the present embodiment, for example, the arithmetic processing section 25c may perform processing for generating a new mapping table by replacing the pixel position (PXA, PYA) before rotation, which is specified in the mapping table included in the control information read from the memory 24, with the pixel position (PXB, PYB) after the rotation, which is acquired as described above, and the mapping processing portion 51 may perform mapping processing using the new mapping table. With such a configuration, the rotated image can be generated directly by the mapping processing performed by the mapping processing portion 51, which eliminates the need for the remapping processing by the image processing portion 52. As a result, it is possible to sufficiently ensure the resource of the image processing portion 52 when the image processing portion 52 performs predetermined image processing on the rotated image, for example.

In addition, with the present embodiment, for example, the output of the observation image from the output processing portion 53 to the display device 4 may be suspended in response to the operation of the freeze switch (not shown) of the input device 5.

Note that the present invention is not limited to the above-described embodiment, and it is needless to say that various changes and modifications are possible in a range without departing from the gist of the invention.

What is claimed is:

1. An optical scanning observation system comprising:
a light-guide configured to guide illumination light supplied from a light source unit, and emit the illumination light from an end portion of the light-guide;
an actuator configured to cause the end portion of the light-guide to oscillate, to thereby be capable of shifting, along a spiral-shaped scanning path, an irradiation position of the illumination light emitted to an object through the end portion;
a light detection section configured to detect return light from the object, generate a light detection signal based on the detected return light, and output the generated light detection signal;
an error angle acquisition section configured to perform processing for acquiring an error angle indicating a degree of deviation of the irradiation position of the illumination light, the irradiation position corresponding to an outermost point of the spiral-shaped scanning path; and
an image generation section configured to perform processing for generating a rotated image by rotating pixel information acquired by converting the light detection signal outputted from the light detection section by an angle acquired by subtracting the error angle from a desired angle of rotation with a center point of the spiral-shaped scanning path as a rotation center.

2. The optical scanning observation system according to claim 1, wherein the image generation section performs processing for generating an original image by mapping the pixel information based on a table indicating a correspondence relation between an output timing of the light detection signal and a pixel position as a destination to which the pixel information is applied, and generating the rotated image by rotating the pixel information in each pixel position of the original image by the desired angle of rotation.

3. The optical scanning observation system according to claim 2, further comprising a setting section configured to perform processing for extracting a pixel at a pixel position corresponding to the center point of the spiral-shaped scanning path from the table, and setting the extracted pixel as a pixel of the rotation center of the rotated image generated by the image generation section.

4. The optical scanning observation system according to claim 2, wherein the image generation section performs processing for magnifying or reducing the original image in addition to the processing for generating the rotated image.

5. The optical scanning observation system according to claim 1, wherein the error angle is stored in a memory provided in an endoscope including the light-guide and the actuator.

6. The optical scanning observation system according to claim 1, wherein the image generation section performs operation for causing a display device to display visual information indicating a current set value of the desired angle of rotation together with the rotated image.
